(19) United States
(12) Patent Application Publication
(10) Pub. No.: US 2024/0203535 A1
(43) Pub. Date: Jun. 20, 2024

(54) Targeted Mass Inoculation

(71) Applicant: Ending Patient Zero, LLC, Ponte Vedra Beach, FL (US)

(72) Inventors: Sarah Rose Wicker, Ponte Vedra Beach, FL (US); Mary Kathryn Reynolds, Ponte Vedra Beach, FL (US)

(21) Appl. No.: 18/595,192

(22) Filed: Mar. 4, 2024

Related U.S. Application Data
(63) Continuation-in-part of application No. 18/125,069, filed on Mar. 22, 2023, now Pat. No. 11,944,678.
(60) Provisional application No. 63/322,700, filed on Mar. 23, 2022.

(51) Int. Cl. G16B 50/30; A61K 39/395; C12N 15/85; G16H 10/60; G16H 50/80

(52) U.S. Cl. CPC G16B 50/30; A61K 39/395; C12N 15/85; G16H 10/60; G16H 50/80

(57) ABSTRACT

In general, the present disclosure involves targeted mass inoculation, and in particular, intelligent systems for mass-inoculating a plurality of hosts without delivering multiple doses to any single individual. In an illustrative example, a transmission system for targeted mass inoculation includes a first pathogen, a primary host that receives the first pathogen, a vector that receives a second pathogen from the primary host, and a plurality of secondary hosts each configured to receive a single dose of the second pathogen from the vector. In some such implementations, the vector includes a mechanical device having internal processing circuitry configured to distinguish between different secondary hosts, and maintain a database indicating which of the plurality of secondary hosts have already received the single dose of the second pathogen.

### CROSS-REFERENCE TO RELATED APPLICATION

This application claims priority to and the full benefit of U.S. Nonprovisional patent application Ser. No. 18/125,069 (filed Mar. 22, 2023, and titled “SYSTEM AND METHODS FOR TARGETED MASS INOCULATION”), the entire contents of which are incorporated in this application by reference, which claimed priority to and the full benefit of U.S. Provisional Patent Application Ser. No. 63/322,700 (filed Mar. 23, 2022, and titled “SYSTEM AND METHODS FOR TARGETED MASS INOCULATION”), the entire contents of which are incorporated in this application by reference.

### BACKGROUND

Since mankind's beginning, the challenge of infectious disease has persisted. Archaeological studies have shown that even 4,000 years ago, people attempted to fight against infection with every resource available. Unfortunately, even today, the causative agents of diseases, including viruses, parasites, bacteria, and fungi, have extraordinarily varied life cycles and modes of transmission. These diseases have the ability to adapt and persist not only in humans, but other animal species as well.

Many disease-causing agents have silent transmission cycles involving wild animals that co-evolve with the infectious agent and exhibit no symptoms of infection. Some diseases occur when a causative agent harbored by a wild animal jumps species to domesticated animals and thence to humans. Others are primarily diseases of domesticated animal species. Humans may be infected by direct contact with wild or domesticated animals, or indirectly by ingestion of contaminated milk or meat, inhalation of aerosolized secretions or excrement, or by being bitten by insects or ticks.

Despite this complexity of epidemiological patterns, the opportunities for intervention often boil down to a few simple bottlenecks in the disease-transmission process. For example, milk-borne diseases can be prevented by pasteurization, certain meat-borne diseases by inspection and animal-husbandry improvements, and other diseases avoided by limiting contact with known high-risk species.

Where the risk of infection is high, or the resulting disease severe, vaccines may be the most efficient and cost-effective means of disease prevention and control. Alternative methods for control of diseases have generally employed trapping, poisoning, or other means of destroying the offending animal vector; however, these methods have mixed (and often negative) records of success and are becoming socially unacceptable.

Vaccines have taken many forms to become effective at disease prevention. In addition to human vaccination, animals that are traditional hosts for diseases have also been immunized. Different methods of immunization have been explored in efforts to effectuate widespread immunization quickly. For example, to increase the rate of dissemination, carriers such as mosquitoes have been investigated as potential vaccine distributors. However, using an autonomous, living organism to vaccinate others present numerous ethical and practical problems that render it an unfeasible alternative.

One challenge with using carriers is controlling the number of vaccinations each animal or person would receive. For instance, one study placed several mice in a container with mosquitoes carrying a vaccine and discovered the number of bites each mouse received varied greatly. Such a large variation in the number of mosquito bites results in vastly different doses of the vaccine being administered. To illustrate this conundrum, it would be similar to administering one dose of a measles vaccine to some people while administering 500 doses to others. Additionally, the use of mosquitoes to vaccinate people would inevitably result in vaccinating at least some people without their informed consent.

### SUMMARY OF THE DISCLOSURE

What is needed are systems and methods for targeted mass inoculation. Such systems and methods may provide for efficient and effective ways of preventing the spread of one or more diseases in a variety of populations, such as human, animal, and insect. In some aspects, systems and methods for targeted mass inoculation may comprise one or more of a plurality of different vectors to aid in the distribution of at least one pathogen. When a vector includes a live organism, such as a mosquito, a method is needed to overcome dosage-distribution issues and ensure the pathogen is administered to an intended secondary host.

The present disclosure provides for systems and methods for targeted mass inoculation. In some embodiments, a transmission system for targeted mass inoculation may comprise a first pathogen and a second pathogen. In some implementations, the first pathogen may comprise a vaccination for an infectious disease. In some implementations, the second pathogen may comprise one or more antibodies. In some aspects, the transmission system may comprise at least one host that receives the first pathogen. In some embodiments, the transmission system may comprise at least one vector that receives the second pathogen from the host. In some implementations, the second pathogen may inoculate a secondary host when the secondary host receives the second pathogen from the vector. In some aspects, at least one portion of the first pathogen may be at least partially different from at least one portion of the second pathogen. In some non-limiting exemplary embodiments, the first pathogen may be the same as the second pathogen.

In some aspects, the vector may comprise at least one mechanical device. In some implementations, a vector that comprises a mechanical device may be configured to deliver at least one of: a first pathogen and a second pathogen to a secondary host. In some embodiments, when the vector comprises a mechanical device, the vector may further comprise at least one computing device. In some implementations, the computing device may identify at least one suitable secondary host. In some aspects, the computing device may comprise at least one algorithm for facilitating secondary host recognition and differentiation by the computing device. In some implementations, the computing device may retain or be communicatively coupled to at least one database comprising one or more indicators of which secondary-host candidates have already received the first or second pathogen.

### BRIEF DESCRIPTION OF THE DRAWINGS

The accompanying drawings that are incorporated in and constitute a part of this specification, illustrate several embodiments of the disclosure and, together with their descriptions, serve to explain the principles of the disclosure.

FIG. 1 illustrates an exemplary transmission system, according to some embodiments of the present disclosure.

FIG. 2 illustrates an exemplary transmission system, according to some embodiments of the present disclosure.

FIG. 3 illustrates an exemplary transmission system comprising immunization, according to some embodiments of the present disclosure.

FIG. 4A illustrates an exemplary transmission system, according to some embodiments of the present disclosure.

FIG. 4B illustrates an exemplary transmission system, according to some embodiments of the present disclosure.

FIG. 5 illustrates an exemplary algorithm, according to some embodiments of the present disclosure.

FIG. 6 illustrates an exemplary algorithm, according to some embodiments of the present disclosure.

FIG. 7 illustrates exemplary method steps for a process for targeted mass inoculation using a transmission system, according to some embodiments of the present disclosure.

FIG. 8 illustrates exemplary method steps for a process for targeted mass inoculation using a transmission system, according to some embodiments of the present disclosure.

FIG. 9 illustrates a conceptual diagram of an example immunization system, according to some embodiments of the present disclosure.

FIG. 10 illustrates a conceptual diagram of an example immunization system, according to some embodiments of the present disclosure.

The FIGURES of the drawings are not necessarily drawn to scale, as their dimensions can be varied considerably without departing from the scope of the present disclosure.

### DETAILED DESCRIPTION

The present disclosure provides for systems and methods for targeted mass inoculation. In some embodiments, the transmission system includes at least two pathogens. In some implementations, a first pathogen at least partially includes a vaccination for an infectious disease, and a second pathogen at least partially comprise one or more antibodies. In some aspects, the transmission system may comprise at least one host that receives the first pathogen. In some embodiments, the transmission system may comprise at least one vector that receives the second pathogen from the host. In some implementations, the second pathogen may inoculate a secondary host when the secondary host receives the second pathogen from the vector. In some aspects, at least one portion of the first pathogen may be at least partially different from at least one portion of the second pathogen. In some non-limiting exemplary embodiments, the first pathogen may be the same as the second pathogen.

According to some aspects of the present disclosure, mass inoculation through the prescribed methods and systems may facilitate safe administration of a first or second pathogen to a plurality of targeted secondary hosts that may prevent subsequent human or animal contraction of one or more infectious diseases.

In the following sections, detailed descriptions of examples and methods of the disclosure will be given. The descriptions of both preferred and alternative examples, though thorough, are exemplary only, and it is understood to those skilled in the art that variations, modifications, and alterations may be apparent. It is therefore to be understood that the examples do not limit the broadness of the aspects of the underlying disclosure as defined by the claims.

### Glossary

Vector: as used herein, refers to a carrier of at least one pathogen. In some embodiments, a vector may comprise at least one living organism that delivers the pathogen to at least one intended secondary host upon contracting the pathogen from contact with at least one infected host. In some implementations, a vector may comprise at least one mechanical device that administers the pathogen to a secondary host based on one or more instructions or directions received from embedded software and at least one associated algorithm.
Pathogen: as used herein, refers to an infectious disease, malady, or one or more agents or elements associated therewith. In some aspects, a pathogen may comprise one or more living or dead components. In some embodiments, a pathogen may comprise one or more antibodies. In some implementations, a pathogen may comprise at least one vaccine. In some aspects, a pathogen may comprise a hybrid disease that may further comprise one or more viruses, parasites, bacteria, or fungi, as non-limiting examples, as well as any combination thereof. In some embodiments, a hybrid disease may enable a secondary host to develop one or more antibodies to a second disease based on one or more causative agents of the second disease having a similar structural composition to other previously-administered vaccines.
Host: as used herein, refers to any entity configured to receive at least one pathogen via external administration.
Secondary host: as used herein, refers to a second host who receives at least one pathogen from at least one vector. This is opposed to an original host, which may contract a pathogen via external administration. In some embodiments, a secondary host may comprise at least one intermediary animal that frequently stores viral strains without suffering symptoms. In some aspects, a pathogen may be transmitted to a secondary host to prevent the contraction and subsequent transmission or mutation of one or more diseases. By preventing a secondary host from contracting the pathogen via immunity, subsequent infection of humans through contact, consumption, or other interaction with the secondary host may be substantially prevented.

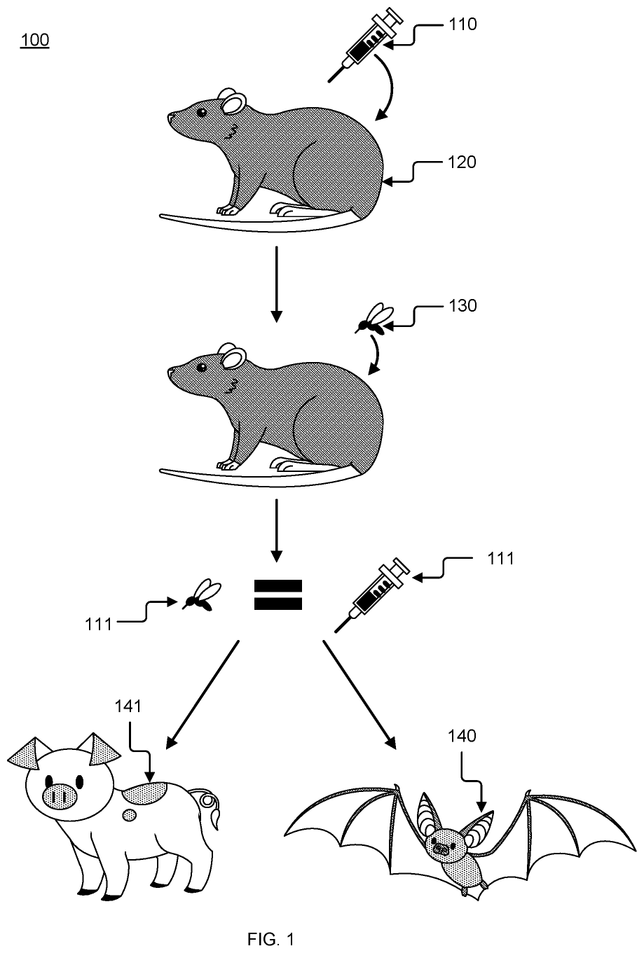

Referring now to FIG. 1, an exemplary transmission system 100, according to some embodiments of the present disclosure, is illustrated. In some embodiments, the transmission system 100 may comprise a first at least one pathogen 110. In some implementations, the transmission system 100 may comprise at least one host 120. By way of example and not limitation, a host 120 may comprise a living organism, such as an insect, animal, bacterium, or human. In some embodiments, the host 120 may contract the first pathogen 110, which may allow for an initial immune response in the host 120. In some aspects, the contraction of the first pathogen 110 may occur via one or more methods, such as injection or ingestion, as non-limiting examples. In some aspects, at least one vector 130 may receive a second at least one pathogen 111 from the host 120. In some embodiments, the vector 130 may transfer the second pathogen 111 to at least one secondary host 140, 141. By way of example and not limitation, a secondary host 140, 141 may comprise at least one living organism, such as a human, animal, or insect.

In some implementations, the first pathogen 110 may at least partially comprise at least one vaccine. In some aspects, the vaccine may comprise one or more portions of the first pathogen 110 that may be insufficient to adversely affect the host 120, but sufficient for the host 120 to develop or produce one or more antibodies to the first pathogen 110. In some embodiments, the vector 130 may receive a second pathogen 111 from the host 120. In some aspects, the second pathogen 111 may at least partially comprise the antibodies developed by the host 120 in reaction to the first pathogen 110. In some implementations, the vector 130 may deliver the second pathogen 111 to at least one secondary host 140, 141.

In some aspects, the secondary host 140, 141 may receive the second pathogen 111 from the vector 130 via digestion or ingestion. For example, a fly or mosquito, acting as a vector 130, may carry the second pathogen 111 and, upon being eaten by a bat, acting as a secondary host, the bat may contract the second pathogen 111 via digestion. In some embodiments, the secondary host 140 may receive the second pathogen 111 from the vector 130 when the vector 130 interacts with the secondary host 140, such as when a mosquito bites a secondary host 140, 141.

By using the vector 130 to distribute the second pathogen 111 to a secondary host 140, the vector 130 may introduce the secondary host to a non-lethal dose of the second pathogen 111. In some aspects, this may allow a secondary host 140, 141 to develop one or more antibodies to the second pathogen 111 without the risk of developing symptoms in reaction to the second pathogen 111. Because the vector 130 is used to distribute the second pathogen 111 to an animal secondary host 140, 141, there may be no need for consent that may be required to inoculate humans.

In some implementations, the second pathogen 111 may comprise sufficient strength to make the secondary host 140, 141 resistant to the second pathogen 111, but not of sufficient potency to cause harm to the secondary host 140, 141 if the secondary host 140, 141 receives more than one dosage of the second pathogen 111, such as when more than one mosquito may bite the same pig, as a non-limiting example. In some embodiments, the vector 130 may comprise at least one genetic modification sufficient to render the vector 130 unable to reproduce, thereby facilitating at least partial control over distribution of the second pathogen 111.

In some aspects, the first pathogen 110 may comprise a hybrid bacterium or virus that utilizes traditional infiltration methods to enter a body and provide immunization. As an illustrative example, this hybrid formation may involve replacing the gene encoding the yellow fever vaccine virus' envelope protein with the corresponding gene of West Nile virus. The hybridization process may comprise replacing the gene encoding the yellow fever vaccine virus' envelope (E) protein with the corresponding gene of West Nile virus. The hybrid formation with the E gene may comprise a plurality of attenuating mutations because of the hybridization.

This hybrid vaccine may enable a host-restricted vaccine to adapt to a secondary host 140, 141. In some aspects, a secondary host 140, 141 inoculated with the hybrid vaccine may develop one or more neutralizing antibodies against West Nile and become protected against future severe intrathecal challenges that may manifest as a result of a West Nile virus infection.

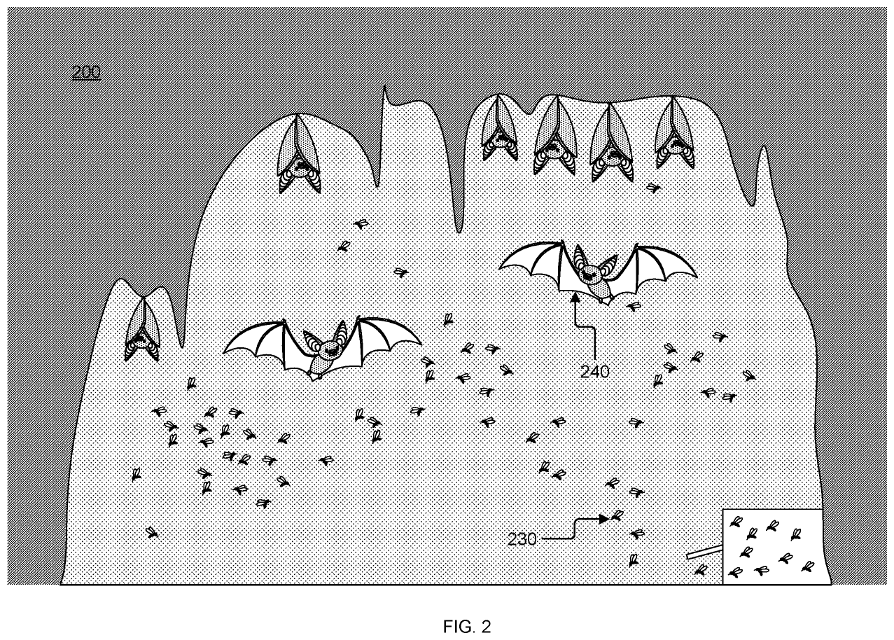

Referring now to FIG. 2, an exemplary transmission system 200, according to some embodiments of the present disclosure, is illustrated. In some embodiments, the transmission system 200 may comprise a plurality of vectors 230. In some implementations, the vectors 230 may interface with a plurality of secondary hosts 240, wherein the interfacing may comprise the vectors 230 interact with secondary hosts 240, such as, for example and not limitation, by biting the secondary hosts 240 or by being consumed by the secondary hosts 240.

In some aspects, the vectors 230 may transfer at least one pathogen to the secondary hosts 240. In some embodiments, the pathogen may allow the secondary hosts 240 to form one or more antibodies that may make the secondary hosts 240 resistant to future contraction of the pathogen. In some implementations, the vectors 230 may be released in a concentrated or confined area to provide intentional interaction with the intended secondary hosts 240. In some embodiments, the vectors 230 may be released when the system determines that the next inoculation is due in accordance with non-limiting factors, such as time, date, or season. In some implementations, the vectors 230 may be released based on schedules in accordance with non-limiting factors, such as time, date or season. In some aspects, the vectors 230 may be released in accordance with one or more predetermined factors, such as time, date, season, weather conditions, or any other factor(s) that may potentially facilitate the interaction between the vectors 230 and the secondary hosts 240.

As an illustrative example, a group of non-reproductive mosquitos may be released at or near the entrance of a cavern that a large population of bats are known to inhabit. A concentrated number of mosquitoes in a confined area may minimize or prevent any accidental interaction and infection of other unintended hosts by the mosquitos. In some aspects, the mosquitos may carry at least one pathogen contracted from at least one previous animal that may spread to the bats upon contact. In some embodiments, the mosquitos may be live bait that distribute the pathogen to the bats upon digestion.

In some implementations, the pathogen may be transferred as the mosquitoes bite the bats. In some aspects, the pathogen may be nonlethal to the bats. For example, the virus may be modified to remove the lethality through manipulation of the genes and proteins of said virus. In some implementations, the bats may, upon contracting the pathogen, develop or produce one or more antibodies to the contracted pathogen. These antibodies may allow the bats to resist future contraction of the same pathogen. Even though the pathogen may be harmless to the bats, the resistance of the bats to the pathogen may prevent the pathogen from being passed onto creatures that may have future interactions with the bats, such as humans. In some aspects, humans may be more susceptible to the pathogen, and by nature of the bat's resistance, humans may not contract a pathogen that may normally be transferred to humans via direct contact with bats.

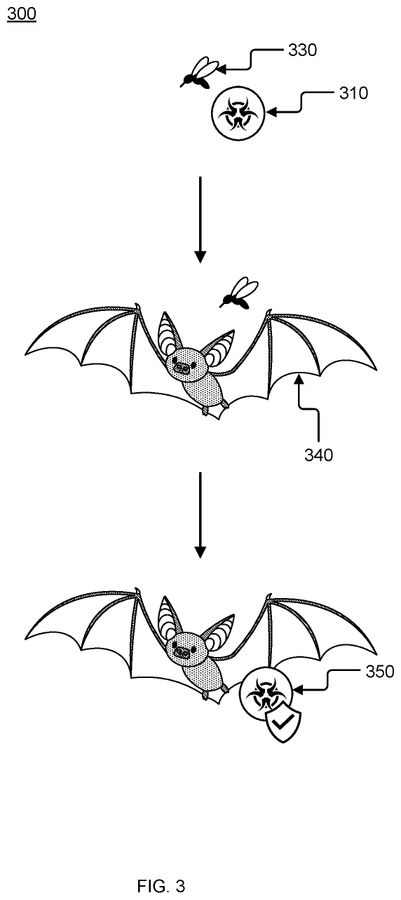

Referring now to FIG. 3, an exemplary transmission-and-immunization system 300 comprising immunization, according to some embodiments of the present disclosure, is illustrated. In some embodiments, at least one vector 330 may transfer at least one pathogen 310 to at least one secondary host 340. In some implementations, the pathogen 310 may provide the secondary host with immunization 350.

In some aspects, the pathogen 310 may comprise at least one hybrid bacterium or virus that utilizes traditional infiltration methods to enter a body and provide immunization 350. As an illustrative example, because the West Nile virus and the Yokose virus may be both of the Flavivirus genus, an existing yellow fever vaccine, or one similar to it, may be incorporated into the development of a Yokose vaccine. This hybrid formation may involve replacing the gene encoding the yellow fever vaccine virus' envelope protein with the corresponding gene of the Yokose virus.

This hybrid vaccine may enable a host-restricted vaccine to adapt to a secondary host 340. In some aspects, a secondary host 340 inoculated with the hybrid vaccine may develop one or more neutralizing antibodies against the Yokose virus and thereby become protected against future severe intrathecal challenges.

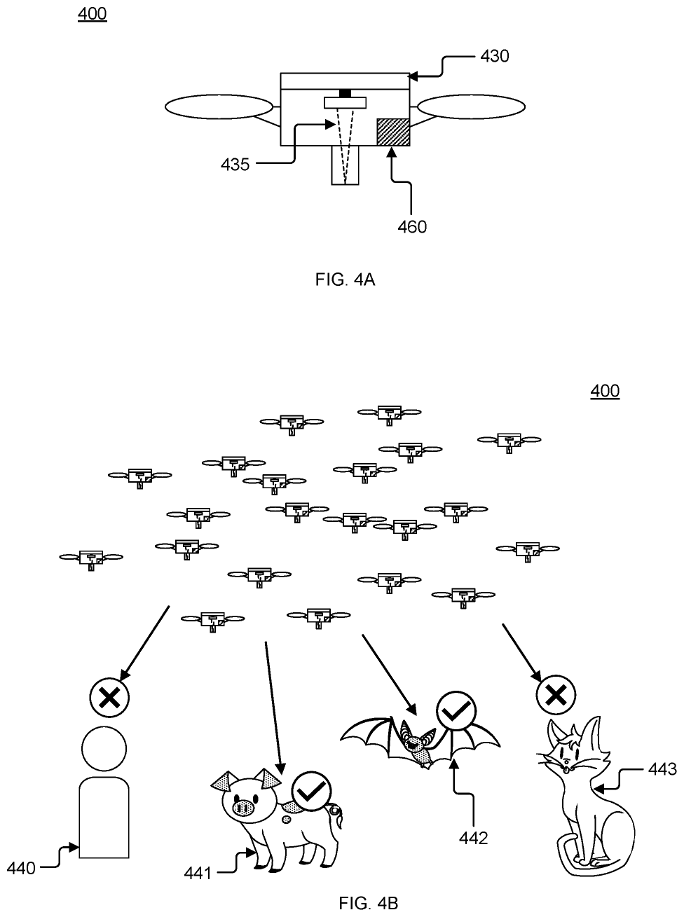

Referring now to FIGS. 4A-B an exemplary transmission system 400 that implements, according to some embodiments of the present disclosure, is illustrated. In some embodiments, the transmission system 400 may comprise at least one vector 430. In some aspects, the vector 430 may comprise at least one mechanical device, such as, by way of example and not limitation, an unmanned vehicle, which may be aerial, terrestrial, or aquatic. In some implementations, the vector 430 may comprise at least one injection device 435, such as a needle or syringe, as non-limiting examples. In some aspects, the vector 430 may immunize or inoculate at least one preferred, predetermined, or targeted secondary host 441, 442. In some aspects, a preferred secondary host 441, 442 may be determined using one or more predetermined variables that may be associated with secondary hosts 440, 441, 442, 443.

By way of example and not limitation, the predetermined variables may comprise at least one of: an age or age range, gender, breed, or subspecies, prior immunization or previous pathogen reception status, species, or location of the secondary host 440, 441, 442, 443. For example, a vector 430 may be configured to contact a preferred secondary host 441, 442 that comprises a species as well as an age or age range that may be safe for contracting at least one pathogen. In some non-limiting exemplary embodiments, a vector 430 may be configured to contact a preferred secondary host 441, 442 that is an animal, such as a pig or a bat.

By way of example and not limitation, a vector 430 may be configured to recognize whether a secondary host 440, 441, 442, 443 is within a predetermined geographical location, such as by utilizing one or more Global Positioning System (GPS) sensors. By way of further example and not limitation, a vector 430 may be configured to recognize whether a secondary host 440, 441, 442, 443 has already received or contracted a pathogen, such as by using one or more infrared sensors that may detect an infrared signature of the secondary host 440, 441, 442, 443, which, in some instances, may be raised or elevated when the secondary host 440, 441, 442, 443 is experiencing an immune response to the pathogen.

In some embodiments, the vector 430 may comprise at least one mechanical device. In some implementations, the vector 430 may comprise at least one computing device 460. In some aspects, the computing device 460 may comprise one or more executable algorithms. In some embodiments, the algorithms may be configured to differentiate between preferred, predetermined, or targeted secondary hosts 441, 442 and restricted secondary hosts 440, 443, wherein the preferred secondary hosts 441, 442 may be determined using one or more predetermined variables. By way of example and not limitation, the predetermined variables may comprise at least one of: an age or age range, gender, breed, or subspecies, prior immunization or previous pathogen reception status, species, or location of the secondary host 440, 441, 442, 443.

In some aspects, the vector 430 may comprise one or more sensors, wherein the sensors may be configured to determine one or more predetermined factors of the secondary hosts 440, 441, 442, 443. By way of example and not limitation, sensors may comprise one or more of: temperature sensors, location sensors, heat sensors, infrared sensors, proximity sensors, motion sensors, light sensors, presence sensors, or sound sensors. In some embodiments, the vector 430 may comprise at least one transmitting device, wherein the transmitting device may be configured to send at least one datum obtained by the sensors to the computing device 460 or to at least one external or remote server. In some implementations, the vector 430 may comprise at least one storage medium, such as, for example and not limitation, a database.

In some implementations, the computing device 460 may be configured to detect, identify, and/or generate one or more differentiation markers to indicate which preferred secondary hosts 441, 442 have previously received or contracted a pathogen. In some embodiments, the differentiation markers may facilitate prevention of multiple injections to preferred secondary hosts 441, 442 that have already received or contracted the pathogen by instructing the computing device 460 to refrain from delivering the pathogen to preferred secondary hosts 441, 442 that have already received the pathogen.

In some aspects, the differentiation markers may comprise one or more physical markings or indicators located directly on the preferred secondary hosts 441, 442 or one or more virtual markings that may correspond to one or more identifiers within at least one database. In some implementations, the computing device 460 may be configured with artificial intelligence. In some aspects, the artificial intelligence of the computing device 460 may facilitate detection or identification of the differentiation markers. In some non-limiting exemplary embodiments, a computing device 460 that comprises artificial intelligence may be trained using at least one machine learning process. By way of example and not limitation, a machine learning process for the computing device 460 may comprise iteratively presenting the computing device 460 with different images of one or more preferred hosts 441, 442, some of which comprise one or more differentiation markers, so that the computing device 460 may progressively improve its ability to detect and identify differentiation markers on preferred secondary hosts 441, 442.

In some non-limiting exemplary embodiments, the vector 430 may be configured to apply one or more differentiation markers that comprise physical markings or indicators to a preferred secondary host 441, 442 once the preferred secondary host 441, 442 has received or contracted a pathogen. By way of example and not limitation, the vector 430 may comprise one or more dispensing mechanisms, such as a nozzle, sprayer, or similar applicator that may dispense at least one substance that may be applied to the preferred secondary host 441, 442 after the preferred secondary host 441, 442 receives or contracts a pathogen.

As an illustrative example, the vector 430 may dispense a substance in the form of dye or paint that may at least temporarily adhere to at least one external portion of the preferred secondary host 441, 442 to form at least one physical marking such that the marking may be subsequently recognized by the computing device 460 using at least one visual capture device, such as a camera or scanner, so that the computing device 460 may determine that the preferred secondary host 441, 442 has previously received or contracted the pathogen, thereby causing the computing device 460 to refrain from delivering the pathogen to that preferred secondary host 441, 442 in the future. In some aspects, one or more vectors 430 may be configured to apply differentiation markers to a plurality of preferred secondary hosts 441, 442 simultaneously, such as when the plurality of preferred secondary hosts 441, 442 may receive a pathogen contemporaneously, such as during a mass inoculation process that may comprise the dispersion of pathogens in the form of aerosolized vapor, as a non-limiting example.

In some aspects, the vector 430 may apply one or more substances to one or more external portions of a preferred secondary host 441, 442 that may reflect wavelengths of a portion of the electromagnetic spectrum that are not perceivable by the human eye, but that may be detectable by the computing device 460 using one or more sensors, such as, for example and not limitation, an infrared or ultraviolet sensor or an infrared or ultraviolet camera. In some non-limiting exemplary embodiments, the vector 430 may use an injection device 435 to inject a preferred secondary host 441, 442 with one or more substances that may be detectable within the preferred secondary host 441, 442 from outside the preferred secondary host 441, 442 by the computing device 460 using one or more scanning devices, such as those used to perform x-radiation imaging or ultrasonography, as non-limiting examples.

In some non-limiting exemplary embodiments, one or more preferred secondary hosts 441, 442 may be physically tagged or branded when they have received the pathogen, either manually or via the vector 430, or the computing device 460 may identify one or more unique physical characteristics, such as, for example and not limitation, fur or skin patterns or facial features, of preferred secondary hosts 441, 442 that have received the pathogen, wherein the unique characteristics may be stored as identifiers in at least one database or other storage medium and then subsequently referenced to identify the preferred secondary hosts 441, 442 at a later time. In some aspects, the computing device 460 may comprise visual recognition or other identifying software to confirm only intended preferred secondary hosts 441, 442 receive the pathogen.

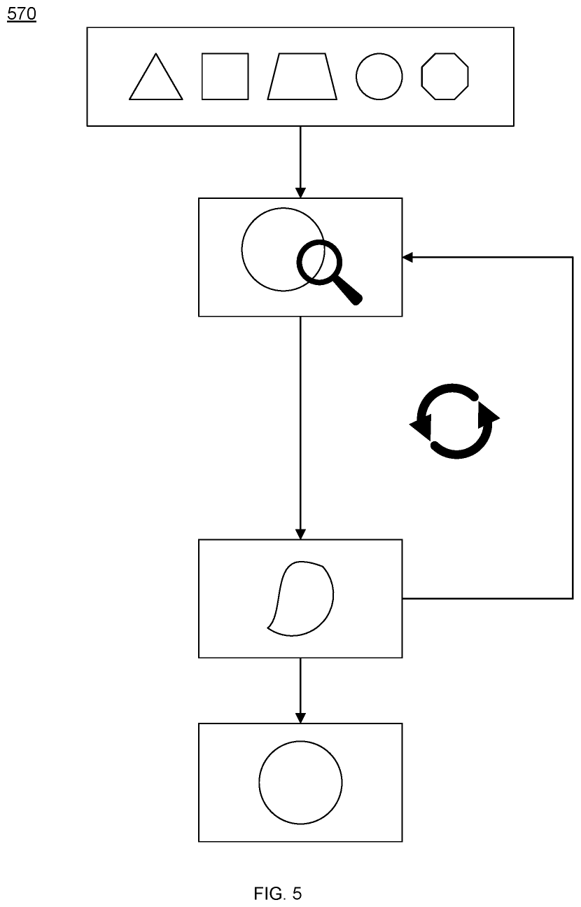

Referring now to FIG. 5, an exemplary algorithm 570, according to some embodiments of the present disclosure, is illustrated. In some embodiments, the algorithm 570 may receive a plurality of predetermined variables. In some implementations, the algorithm 570 may group predetermined variables by one or more predefined rules, associations, or attributes, as a non-limiting list.

In some embodiments, the algorithm 570 may receive a plurality of predetermined variables. In some aspects, the algorithm 570 may comprise machine learning. In some implementations, the algorithm 570 may utilize machine learning to reference the predetermined variables for comparison to at least one submitted identification. In some aspects, the algorithm 570 may utilize one or more identified attributes associated with the predetermined variables to classify the submitted identification. In some embodiments, the algorithm 570 may use machine learning to provide a ranking of similarity between the submitted identification and the received predetermined variables. In some implementations, the algorithm 570 may classify the submitted identification and associate the submitted identification with one or more of the predetermined variables.

As an illustrative example, the algorithm 570 may receive one or more images of a pig for the purpose of visual recognition of pigs. The pictures of pigs may comprise pigs with various skin colors and patterns. The algorithm 570 may use the colors, patterns, and other non-limiting variables common among the pictures of pigs to further categorize future visually inspected pigs.

In some aspects, the algorithm 570 may use identified attributes to classify an animal. For example, the algorithm 570 may notice that an animal comprises a relatively small size and has a body, a head with pointed ears, four legs, a long tail, and fur. Without additional facts, the algorithm 570 may use the combination of identified attributes of the observed animal to conclude that the animal is a cat. In some implementations, the algorithm 570 may indicate a low association between the submitted identification and the received predetermined variables, thereby indicating that the cat is not likely to be a pig. In some aspects, the algorithm 570 may use a submitted identification to generate one or more new predetermined variables.

In some embodiments, the algorithm 570 may comprise one or more recursive functions that may improve accurate identification over time. In some implementations, the algorithm 570 may quantify accurate identification by a predetermined threshold of acceptance or accuracy. In some aspects, the algorithm 570 may improve over time by using the data collected and categorized from previous animals to improve the algorithm's likelihood for correctly identifying animals in the future.

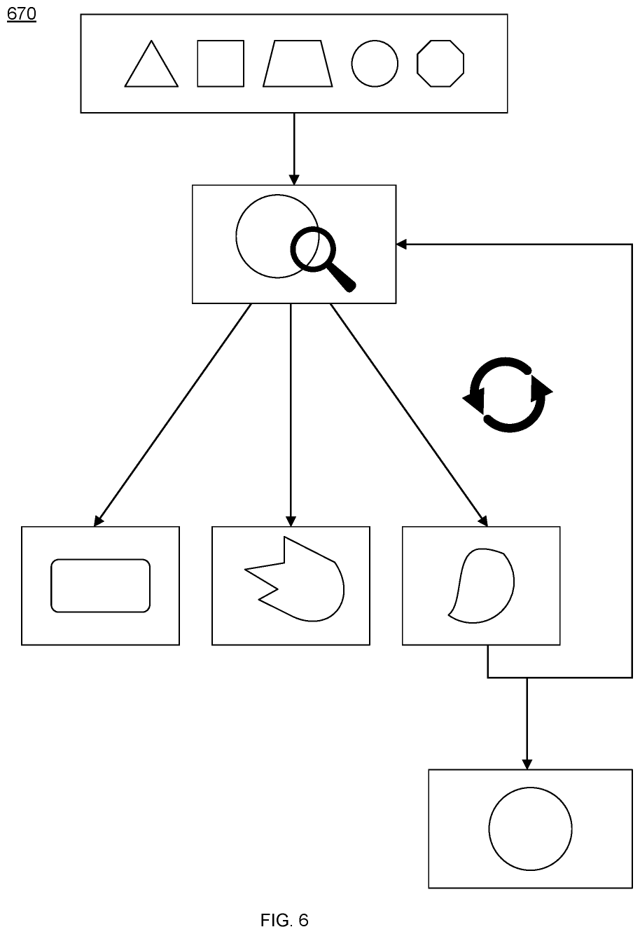

Referring now to FIG. 6, an exemplary algorithm 670, according to some embodiments of the present disclosure, is illustrated. In some embodiments, the algorithm 670 may receive a plurality of predetermined variables. In some implementations, the algorithm 670 may reference the predetermined variables for comparison to a submitted identification. In some aspects, the algorithm 670 may utilize one or more identified attributes associated with the predetermined variables to classify the submitted identification.

In some embodiments, the algorithm 670 may provide a ranking of similarity between the submitted identification and the received predetermined variables. In some implementations, the algorithm 670 may classify the submitted identification and associate the submitted identification with one or more of the predetermined variables. In some embodiments, the algorithm 670 may provide one or more classifications for a submitted identification that may associate the submitted identification with two or more of the predetermined variables. In some aspects, the algorithm 670 may configure artificial intelligence to discover trends in the inputted data. In some embodiments, the algorithm 670 may use clustering, dimensionality reduction, and decision tree classifiers to sort the trends discovered by artificial intelligence into complex groupings. In some aspects, the algorithm 670 may produce classifications for two or more submitted identifications simultaneously.

In some implementations, the algorithm 670 may indicate a low association between the submitted identification and the received predetermined variables. In some aspects, the algorithm 670 may use a submitted identification to generate one or more new predetermined variables. In some embodiments, the algorithm 670 may refine new predetermined variables recursively based on feedback from subsequently submitted identifications. In some implementations, new predetermined variables may become distinct from other received predetermined variables via recursion of the algorithm 670.

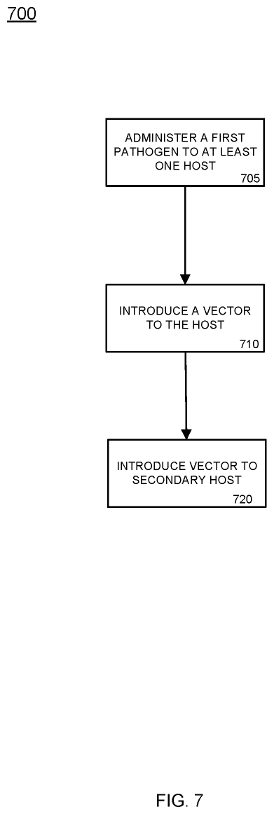

Referring now to FIG. 7, exemplary method steps for a process 700 for targeted mass inoculation using a transmission system, according to some embodiments of the present disclosure, are illustrated. In some aspects, at 705, the process 700 may comprise administering a first at least one pathogen to at least one host. In some embodiments, the first pathogen may be transmitted to the host via injection, aerosolized vapor, or consumption, as non-limiting examples of pathogen transmission. In some implementations, at 710, at least one vector may be introduced to the host, wherein the introduction may facilitate transfer of a second at least one pathogen from the at least one host to the at least one vector.

By way of example and not limitation, a plurality of vectors that comprise mosquitos may be released into a confined area with a host that comprises a mouse that has received the first pathogen. In some embodiments, the vector may extract a second pathogen from the host that comprises at least one portion of a vaccine or one or more antibodies that may be transferred to at least one secondary host. In some implementations, there may be a predetermined amount of time allotted between the host receiving the first pathogen and the vector extracting the second pathogen to ensure maximum potency of the extracted second pathogen from the host. In some non-limiting exemplary embodiments, the vector may comprise at least one computing device configured to determine when the second pathogen is receivable from the host, such as, for example and not limitation, by determining when a predetermined amount of time has elapsed after the host has received the first pathogen.

In some aspects, at 720, the process 700 may comprise introducing the vector to at least one secondary host, wherein the introduction may facilitate insertion of the second pathogen, via the vector, into the secondary host. In some embodiments, the secondary host may reside in a confined space. In some implementations, a plurality of vectors may comprise two or more living organisms, such as a swarm of infected mosquitoes. In some aspects, the vectors may be rendered incapable of reproducing and released in a concentrated quantity into an open environment where a plurality of secondary hosts reside.

In some embodiments, the vector may administer the second pathogen by biting the secondary host or by being consumed by the secondary host, as non-limiting examples. In some aspects, the second pathogen may be administered to the secondary host to prevent the contraction and subsequent transmission or mutation of one or more diseases. In some implementations, by initiating an immune response within the secondary host, the secondary host may develop immunity to the second pathogen, such that subsequent infection of humans through contact, consumption, or other interaction with the secondary host may be substantially prevented.

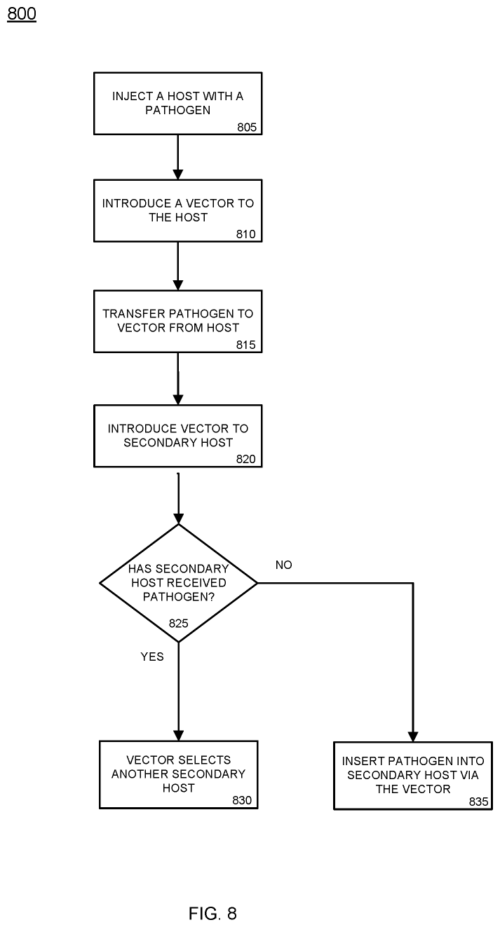

Referring now to FIG. 8, exemplary method steps for a process 800 for targeted mass inoculation using a transmission system, according to some embodiments of the present disclosure, are illustrated. In some aspects, at 805, at least one host may receive a first at least one pathogen via, for example and not limitation, external injection. In some non-limiting exemplary embodiments, the first pathogen may be transmitted to the host via aerosolized vapor or consumption, as further non-limiting examples of pathogen transmission.

In some implementations, at 810, at least one vector may be introduced to the host with the intention of contracting or otherwise receiving a second at least one pathogen, wherein the second pathogen may be the same as the first pathogen, or the second pathogen may comprise one or more at least partially different aspects from the first pathogen. By way of example and not limitation, the second pathogen may comprise one or more antibodies developed by the host in response to the first pathogen. By way of further example and not limitation, a plurality of vectors that comprise drone-based mechanical devices may receive a second pathogen from a mouse that received a first pathogen.

In some implementations, at 815, the vector may receive the second pathogen from the host. In some embodiments, the vector may extract a second pathogen that comprises at least one portion of a vaccine or one or more antibodies that may be transferred to at least one secondary host. In some implementations, there may be a predetermined amount of time allotted between the host receiving the first pathogen and the vector extracting the second pathogen to ensure maximum potency of the extracted second pathogen from the host. In some non-limiting exemplary embodiments, the vector may comprise at least one computing device configured to determine when the second pathogen is receivable from the host, such as, for example and not limitation, by determining when a predetermined amount of time has elapsed after the host has received the first pathogen.

In some aspects, at 820, the vector may be introduced to the secondary host. In some embodiments, the secondary host may reside in a confined space. In some implementations, a plurality of vectors may comprise two or more mechanical devices, such as a plurality of drone-based mechanical devices. In some aspects, the vectors may be released in a concentrated quantity into an open environment where a plurality of secondary hosts reside. In some embodiments, the vectors may administer the second pathogen via direct injection or aerosol disbursement, as non-limiting examples.

In some implementations, at 825, it may be determined whether one or more secondary hosts have previously received the second pathogen. In some aspects, this determination may be facilitated by at least one vector that comprises at least one computing device that comprises at least one indication algorithm that enables the vector to differentiate secondary hosts who have already received the second pathogen from those that have not. In some embodiments, if the vector determines that the secondary host has previously received the second pathogen then, at 830, the vector may select an alternative secondary host to which administer the second pathogen.

In some aspects, at 835, the vector may administer the second pathogen to the secondary host. In some embodiments, the vector may administer the second pathogen via an injection device and/or an aerosol as a non-limiting example. In some implementations, the second pathogen may be administered to the secondary host to prevent the contraction and subsequent transmission or mutation of one or more diseases. In some embodiments, by initiating an immune response within the secondary host, the secondary host may develop immunity to the second pathogen, such that subsequent infection of humans through contact, consumption, or other interaction with the secondary host may be substantially prevented.

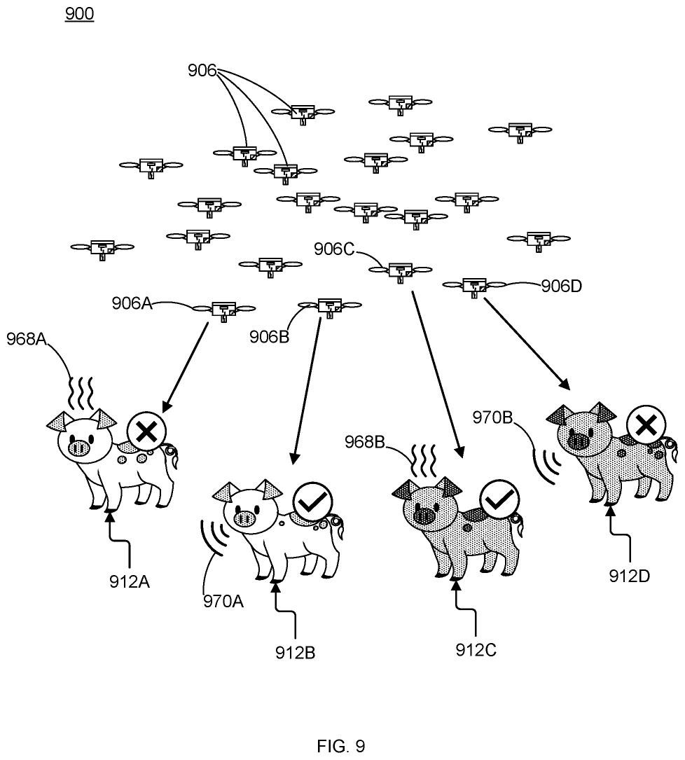

Referring now to FIG. 9, a conceptual diagram of an example immunization system 900, in accordance with the techniques of this disclosure, is illustrated. In some embodiments, system 900 may comprise a plurality of vectors 906 configured to immunize or inoculate a plurality of secondary hosts 912A-912D, without delivering multiple doses of inoculation to any single host 912. In the non-limiting example shown in FIG. 9, secondary hosts 912A-912D may be depicted as animals belonging to the same species. In some aspects, vectors 906 may comprise internal processing circuitry configured to execute AI and/or machine-learning models trained to: (1) distinguish, in real-time, between different secondary hosts 912, even among the same species; (2) determine whether a particular secondary host 912 host has already received a single dose of inoculation; and (3) keep track of which of the secondary hosts 912 have already received a single dose of inoculation.

In some implementations, vector 906A targets secondary host 912A and determines, by analyzing a chemical emission 968A, that secondary host 912A has not yet received a dose of inoculation. In some aspects, a particular signature of pheromone, scent gland or hormone secretion, skin-cell receptors, blood-cell receptors, or other biological marker may have a known correlation with exposure to a certain pathogen, and vector 906A does not detect such marker or signature with respect to secondary host 912A. In some embodiments, in response to determining that secondary host 912A has not yet been vaccinated, vector 906A proceeds to deliver a single dose of the pathogen to secondary host 912A.

In some embodiments, concurrently with delivering the single dose, vector 906A may access a database stored in a non-transitory computer-readable medium, as a non-limiting example, remotely accessible by all of vectors 906 and/or distributed across all of vectors 906 (e.g., using blockchain technology and a decentralized, distributed ledger), and generate a new database entry for host 912A with a “VACCINATED” status, and correlate the new database entry with a set of identifiable characteristics of host 912A (e.g., skin tone, fur-spot pattern, ear shape, pheromone signature, etc.). In some aspects, concurrently with inoculating secondary host 912A, vector 906A may be configured to apply a differentiable marker to secondary host 912A. For instance, vector 906A can simultaneously inject a readable, scannable, or otherwise-detectable tracker into 906A, such as a microscopic tracker “chip.” Additionally or alternatively, vector 906A may apply a visibly identifiable substance to the skin or fur of host 912A.

In some implementations, vector 906B may target secondary host 912B, and queries the shared database to determine whether secondary host 912B has previously been vaccinated. In some embodiments, vector 906B may identify one or more unique characteristics of secondary host 912B, such as skin tone, fur-spot pattern, facial features, ear size and/or shape, eye (e.g., iris) pattern, paw-or-hoof prints, skin-cell receptor signature, blood-cell receptor signature, and/or audible vocalization signature 970A, to identify host 912B within the database. In some aspects, vector 906B may detect an identification chip 912B coupled to or embedded within host 912B, and consult the database entry corresponding to the chip's identifier, to determine whether host 912B has previously been vaccinated. In some embodiments, upon identifying secondary host 912B within the shared database, vector 906B determines from the appropriate database entry that secondary host 912B has already been vaccinated, and in response, does not deliver an additional dose of the second pathogen. In some aspects, vector 906B may then proceed to automatically target a different secondary host.

In some implementations, vector 906C may target secondary host 912C and determines, by analyzing a chemical emission 968B, that secondary host 912C has already received a dose of inoculation. In some aspects, vector 906C may detect a particular signature of pheromone, scent gland, antibody, or other biological marker with a known correlation with having previously encountered a certain pathogen. In some implementations, vector 906C may use an immune test to examine the extent of the immune response to determine whether the secondary host 912C has already been vaccinated. In some embodiments, vector 906C queries the shared database and locates an entry for secondary host 912C based on identifiable characteristics such as skin tone, vocalization pattern, fur-spot pattern, scent, etc. However, in this case, the shared database incorrectly indicates that secondary host 912C is still unvaccinated. In some aspects, vector 906C corrects the database entry to “VACCINATED” (in some cases, only after performing an additional confirmatory analysis of the chemical-emission signature 968B), and leaves to target a different secondary host without delivering additional inoculation to secondary host 912C.

In some embodiments, vector 906D may target secondary host 912D and queries the shared database to determine whether secondary host 912D has previously been vaccinated. In some aspects, vector 906D may identify one or more characteristics of secondary host 912D, such as skin tone, fur-spot pattern, and/or audible vocalization signature 970B, to locate an entry for secondary host 912D within the database. In some embodiments, vector 906D may detect an identification chip 912D coupled to or embedded within host 912D, and consult the database entry corresponding to the chip's identifier, to determine whether host 912D has previously been vaccinated. In some implementations, upon identifying secondary host 912D within the shared database, vector 906D determines from an existing database entry that secondary host 912D has not yet been vaccinated. In response, vector 906D vaccinates secondary host 912D and updates the corresponding database entry to indicate “VACCINATED.” In some aspects, vector 906D may then proceed to automatically target a different secondary host.

In some embodiments, the shared digital memory bank (e.g., of a remote computing device to which all of vectors 906 are communicatively connected), may store a data entry that constitutes a “running tally” of the number of consecutive targeting interactions that do not result in a new inoculation, i.e., where the targeted secondary host may be found to have already been vaccinated. In some aspects, when this tally crosses a certain threshold (e.g., more than 30 consecutive interactions without additional vaccination), system 900 may conclude that all or substantially all of secondary hosts 912 have been vaccinated, and in response, vectors 906 may be configured to automatically stop targeting additional secondary hosts and retreat to a “home base” location for collection.

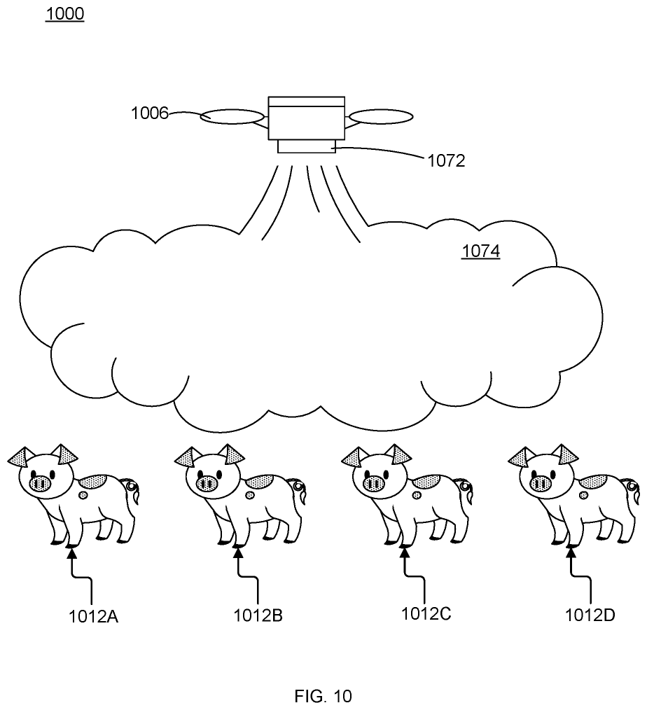

Referring now to FIG. 10, a conceptual diagram of an example immunization system 1000, in accordance with the techniques of this disclosure, is illustrated. In some aspects, system 1000 may comprise one or more vectors 1006 configured to immunize or inoculate a plurality of secondary hosts 1012A-1012D, without delivering multiple doses of inoculation to any single host 1012. In some implementations, vector 1006 may be configured to detect, identify, and/or generate one or more differentiation markers to indicate which of secondary hosts 1012 have previously received or contracted a pathogen, and in response, automatically refrain from delivering additional pathogen to them. In some embodiments, such differentiation markers may comprise physical markings or indicators located directly on the preferred secondary hosts 1012, and/or virtual markings that may correspond to identifiers stored within a shared database. In some implementations, vector 1006 may comprise an integrated computing device (e.g., internal processing circuitry) configured with artificial intelligence (AI) and/or machine-learning models trained to detect and/or identify the differentiation markers.

In some embodiments, vector 1006 may be configured to apply differentiation markers, in the form of physical markings or indicators, to a preferred secondary host 1012 once the preferred secondary host has received or contracted a pathogen. By way of example and not limitation, the vector 1006 may comprise a dispensing mechanism 1072, such as a nozzle, sprayer, or similar applicator, that dispenses a substance 1074 onto secondary host(s) 1012 upon receiving or contracting the second pathogen.

In some implementations, vector 1006 may dispense a substance 1074 in the form of dye or paint that at least temporarily externally adheres to secondary host(s) 1012, thus forming an identifiable physical marking that may be recognized by vectors during subsequent encounters, e.g., using a visual capture device (e.g., a camera or scanner). In some aspects, the vector 1006 may externally apply substances 1074 to secondary hosts 1012 that reflect certain wavelengths of light outside the visible spectrum, but that are detectable by a sensor of vector(s) 1006, such as infrared or ultraviolet-light sensors.

In some embodiments, in response to detection of such markers, vector(s) 1006 refrain from delivering additional pathogen to those secondary hosts 1012 in the future. In some aspects, one or more vectors 1006 may be configured to simultaneously apply such differentiation markers to a plurality of preferred secondary hosts 1012, such as when the plurality of preferred secondary hosts 1012 receive a pathogen contemporaneously, e.g., during a mass-inoculation involving a dispersion of pathogens in the form of aerosolized vapor. In some aspects, vector 1006 may release, via dispensing mechanism 1072: a substance 1074 that may comprise an aerosolized pathogen for inhalation by secondary hosts 1012; an identifiable marker for identifying hosts 1012 that have received a single dose of inoculation; or a mixture of both the aerosolized pathogen and the identifiable marker.

### Conclusion

A number of embodiments of the present disclosure have been described. While this specification contains many specific implementation details, these should not be construed as limitations on the scope of any disclosures or of what may be claimed, but rather as descriptions of features specific to particular embodiments of the present disclosure.

Certain features that are described in this specification in the context of separate embodiments can also be implemented in combination or in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous. Nevertheless, it will be understood that various modifications may be made without departing from the spirit and scope of the claimed disclosure.

What is claimed is:

1. A transmission system for targeted mass inoculation, the system comprising:
a plurality of secondary hosts; and
a vector comprising processing circuitry configured to:
target a first secondary host from among the plurality of secondary hosts;
determine whether the first secondary host has already received a single dose of a pathogen;
in response to determining that the first secondary host has not yet received the single dose of the pathogen, cause the vector to deliver the single dose of the pathogen to the secondary host; and
in response to determining that the first secondary host has already received the single dose of the pathogen, target a second secondary host from among the plurality of secondary hosts without delivering an additional dose of the pathogen to the first secondary host.

2. The system of claim 1, wherein the processing circuitry of the vector is further configured to:
determine an identity of the first secondary host; and
determine whether the first secondary host has already received the single dose of the pathogen based on data associated with the identity of the first secondary host.

3. The system of claim 2, wherein the processing circuitry of the vector is configured to determine the identity of the first secondary host by comparing one or more identifiable characteristics of the first secondary host to a database of entries corresponding to each of the plurality of secondary hosts.

4. The system of claim 3, wherein the one or more identifiable characteristics of the first secondary host comprise:
a skin tone;
a fur pattern;
a vocalization signature;
a chemical-emission signature;
an ear shape; or
an iris signature.

5. The system of claim 3, wherein the system further comprises:
a plurality of vectors comprising the vector; and
a non-transitory computer-readable medium storing the database, wherein each of the plurality of vectors is configured to access the database.

6. The system of claim 5, wherein the plurality of vectors are configured to, in response to delivering the single dose of the pathogen to one of the plurality of secondary hosts, update a vaccination-status field for the respective secondary host within the database.

7. The system of claim 5, wherein the database comprises a decentralized blockchain encoding a current vaccination status for each of the plurality of secondary hosts, and wherein the decentralized blockchain is stored on a distributed ledger within each of the plurality of vectors.

8. The system of claim 5, wherein the non-transitory computer-readable medium further stores a running tally of consecutive targeting interactions between the plurality of vectors and the plurality of secondary hosts that do not result in a subsequent delivery of a single dose of the pathogen to one of the plurality of secondary hosts.

9. The system of claim 8, wherein the plurality of vectors is configured to automatically stop targeting the plurality of secondary hosts when the running tally equals a predetermined threshold.

10. The system of claim 1, wherein the processing circuitry of the vector is configured to determine whether the first secondary host has already received the single dose of the pathogen based on a characteristic of the first secondary host having a known correlation with exposure to the pathogen.

11. The system of claim 10, wherein the characteristic comprises a pheromone signature, a skin-cell-receptor signature, or a blood-cell-receptor signature.

12. The system of claim 1, wherein, in response to determining that the first secondary host has not yet received the single dose of the pathogen, the processing circuitry is further configured to cause the vector to apply an identifiable marker to the first secondary host concurrently with causing the vector to deliver the single dose of the pathogen to the secondary host.

13. The system of claim 12, wherein the processing circuitry is configured to determine that the first secondary host has already received the single dose of the pathogen by detecting the identifiable marker.

14. The system of claim 12, wherein applying the identifiable marker to the first secondary host comprises dispensing a visually identifiable substance onto an exterior of the first secondary host.

15. The system of claim 12, wherein applying the identifiable marker to the secondary host comprises dispensing an aerosolized vapor that further comprises the single dose of the pathogen for delivery to the first secondary host via inhalation.

16. The system of claim 12, wherein the vector is configured to apply the identifiable marker to the first secondary host by injecting a scannable chip into the first secondary host.

17. The system of claim 1, wherein the vector comprises an aerial drone.

18. The system of claim 1, wherein the pathogen comprises a second pathogen, and wherein the system further comprises:
a first pathogen; and
a primary host configured to:
receive the first pathogen via external administration;
produce the second pathogen in response to receiving the first pathogen; and
transmit the second pathogen to the vector.

19. The system of claim 18, wherein the pathogen comprises antibodies produced by the primary host.

20. A method comprising:
targeting, by processing circuitry of a vector, a first secondary host from among a plurality of secondary hosts;
determining, by the processing circuitry of the vector, whether the first secondary host has already received a single dose of a pathogen;
in response to determining that the first secondary host has not yet received the single dose of the pathogen, causing the vector to deliver the single dose of the pathogen to the secondary host; and
in response to determining that the first secondary host has already received the single dose of the pathogen, targeting a second secondary host from among the plurality of secondary hosts without delivering an additional dose of the pathogen to the first secondary host.
